(19) United States
(12) Patent Application Publication
(10) Pub. No.: US 2024/0269054 A1
(43) Pub. Date: Aug. 15, 2024

(54) HYDRATABLE CONCENTRATED SURFACTANT COMPOSITIONS

(71) Applicant: Conopco, Inc., d/b/a UNILEVER, Englewood Cliffs, NJ (US)

(72) Inventors: Nicholas WEBB, Hoylake, Wirral (GB); Aneliya Nikolova ZDRAVKOVA, Liverpool (GB)

(21) Appl. No.: 18/578,443

(22) PCT Filed: Jul. 4, 2022
(86) PCT No.: PCT/EP2022/068481

§ 371 Date: Jan. 11, 2024

(30) Foreign Application Priority Data
Jul. 15, 2021 (EP) 21185950.9

(51) Int. Cl. A61K 8/44; A61K 8/02; A61K 8/34; A61K 8/46; A61Q 5/02

(52) U.S. Cl. CPC A61K 8/442; A61K 8/0295; A61K 8/345; A61K 8/466; A61Q 5/02; A61K 2800/596

(57) ABSTRACT

A hydratable concentrated surfactant composition comprising based on the total weight of the composition and 100% activity: a) from 5 wt % to 40 wt % of a sulphate free anionic surfactant comprising 6 to 22 carbon atoms; 5 b) from 5 wt % to 40 wt % of an amphoteric and/or zwitterionic surfactant; c) from 0.01 wt % to 5 wt % of a first viscosity modifier, selected from electrolytes, polymeric thickeners, ethoxylated fatty acid esters, amines comprising from 12 to 18 carbon atoms and mixtures thereof; d) from 0.1 wt % to 15 wt % of a second viscosity modifier, which is a polyol; 0e) a preservative; and f) from 10 to 70% by weight water; wherein the hydratable concentrated surfactant composition has a pH of from 3 to 6; and wherein the hydratable concentrated surfactant composition has a viscosity of from 6,000 to 400,000 cps, preferably 8000 to 300,000 cps, when measured at 30° C. on a Brookfield DV2T 5using a Spindle RV-05 at 20 rpm for 60 seconds on a Helipath stand.

### FIELD OF THE INVENTION

The present invention is directed to a hydratable concentrated surfactant cleansing composition and a method of making an end use composition by diluting with water. This invention has particular application in the field of personal care, particularly hair care.

### Background and Prior Art

Liquid based cleansing compositions, such as shampoos and body washes, are common and enjoyed by many consumers. Such compositions typically have water as the predominant ingredient, and they are often sold in plastic bottles or tubes. The compositions are conventionally formulated to have a viscosity that is customary for consumer use and easy for evacuation from the package they are sold in.

It is often publicised that the world's oceans will soon contain more plastic than fish. Given environmental concerns and the desire for consumers and conscious companies to do more for the planet, there is a strong desire to use less plastic when selling products, including consumer products. In view of this, efforts have been made to sell product in concentrate form, and therefore, ship product that comprises less water, in smaller packages.

Surfactant containing cleansing products, in the form of concentrates are known in the prior art.

US 2019/031258 A1, describes rheofluidifying concentrated foaming compositions.

US 2018/098923 A1, discloses personal care compositions substantially free of sulfated surfactants.

US 2019/282480 A1, describes self-thickening cleansing compositions with N-acyl acidic amino acids or salts thereof and an amphoteric surfactant.

Concentrates are typically diluted at point of use to form a liquid product that is then usable in the conventional way.

A difficulty encountered with such concentrates is that consumers often do not like adding additional water to the concentrate and having to convert, for example by stirring, the concentrate into a usable end product. As to the hydrated product, common complaints include that the process is time consuming and the resulting product is not homogeneous, and has undesirable viscosity, after adding water.

Indeed, we have found that such concentrated compositions take extended time to dilute properly, prior to use by the consumer and some consumers do not allow enough time for full dilution to take place. It is, therefore, desirable to provide a concentrate that dilutes quickly for easier and more efficient use.

The properties of the diluted concentrate are also important. Consumers strongly prefer thick liquids because they associate thickness with quality, good spreadability and pourability. Ideally, the diluted product of a concentrate would have similar viscosity to a standard format personal care product, such as a shampoo. It is, therefore, desirable to provide a concentrate that produces a diluted product with similar thickness to a conventional liquid product.

It is also desirable to develop a concentrate that is substantially free from sulfate and free from silicone to meet environmental needs.

A formulation that works at acidic pH is also desirable as it allows the use of more naturally derived preservative materials, which typically work at lower pH, for example, sodium benzoate.

We have now surprisingly found that a composition, provided in lamellar crystal phase, is capable of transforming rapidly to an isotropic phase upon dilution. In combination with a high salt content, a thick end dilute can be achieved.

The composition can be used as a concentrate in small volumes and diluted at point of use. It can be diluted with water in refill packaging to ensure a reduction in plastic waste.

### Definition of the Invention

In a first aspect, the invention provides a hydratable concentrated surfactant composition comprising:
a) from 5 wt % to 40 wt % of a sulphate free anionic surfactant comprising 6 to 22 carbon atoms;
b) from 5 wt % to 40 wt % of an amphoteric and/or zwitterionic surfactant;
c) from 0.01 wt % to 5 wt % of a first viscosity modifier, selected from electrolytes, polymeric thickeners, ethoxylated fatty acid esters, amines comprising from 12 to 18 carbon atoms and mixtures thereof;
d) from 0.1 wt % to 15 wt % of a second viscosity modifier, which is a polyol;
e) a preservative; and
f) from 10 wt % to 70 wt % water;
   wherein the hydratable concentrated surfactant composition has a pH of from 3 to 6; and
   wherein the hydratable concentrated surfactant has a viscosity of from 6,000 to 400,000 cps, as measured at 30° C. on a Brookfield DV2T using a Spindle RV-05 at 20 rpm for 60 seconds on a Helipath stand.

In a second aspect, there is provided an end use composition made by hydrating the hydratable concentrated surfactant composition of the first aspect by dilution with water.

Preferably, the end use composition has a weight ratio of hydratable concentrated surfactant composition to water of from 1:1 to 1:6, more preferably 1:1 to 1:5. Preferably, the end use composition has a viscosity of from 2,000 to 10,000 cPs at 30° C., preferably from 2000 to 7,500 cPs, when measured at 30° C. on a Brookfield DV2T using a Spindle RV-05 at 20 rpm for 60 seconds on a Helipath stand.

The hydratable concentrated surfactant composition has a viscosity of from 6,000 to 400,000 cps, preferably from 8000 to 300,000, more preferably from 10,000 to 100,000, even more preferably from 15,000 to 40,000 cps, most preferably, from 15,000 to 30,000 cps.

Upon dilution the viscosity decreases, thus resulting in an end use composition having a viscosity of from 2000 to 10,000 cps, preferably from 2000 to 7,500 cps, as measured at 30° C. on a Brookfield DV2T using a Spindle RV-05 at 20 rpm for 60 seconds on a Helipath stand.

In a third aspect, the present invention provides a method of preparing an end use composition, the method comprising the step of diluting a hydratable concentrated surfactant composition of the first aspect with water. Preferably, the method comprises a step of applying moderate shear such as shaking or stirring to the mixture of hydratable composition and water to yield the end use composition in less than 5 minutes, preferably in less than 3 minutes, more preferably in less than 2 minutes, even more preferably in less than 1 minute and most preferably in less than 30 seconds.

Preferably, the hydratable concentrated surfactant composition is diluted at a hydratable concentrated surfactant composition to water weight ratio of from 1:1 to 1:6, more preferably from 1:1 to 1:6. Preferably, the water has a temperature of from 10 to 50 degrees Celsius.

### DETAILED DESCRIPTION OF THE INVENTION

### Method of Making

When making hydratable concentrated surfactant composition of the present invention, the desired ingredients may be mixed with conventional apparatus under moderate shear and atmospheric conditions, with temperature being from 35 to 80° C.

Water is added to the hydratable concentrated surfactant composition to produce the end use composition. Moderate shear such as shaking (or stirring) in a container will yield the end use composition in less than 5 minutes, preferably in less than 3 minutes, and most preferably, in less than 2 minutes. In an embodiment of the invention, end use composition is made in less than 1 minute, even preferably, less than 30 seconds.

### Viscosities of the Compositions

The hydratable concentrated surfactant composition has a viscosity from 6,000 to 400,000 cps, preferably from 8000 to 300,000 cps, preferably from 15,000 to 40,000 cps, more preferably, from 15,000 to 30,000 cps.

Upon dilution the viscosity increases resulting in an end use composition having a viscosity of from 2000 to 10,000 cps, preferably from 2000 to 7,500 cps, as measured at 30° C. on a Brookfield DV2T using a Spindle RV-05 at 20 rpm for 60 seconds on a Helipath stand.

In the present invention, the hydratable concentrated surfactant composition should be formulated such that upon dilution, the desired component/ingredient levels (such as sulfate levels) in the end use composition are attained.

The hydratable concentrated surfactant composition table composition of the present invention will have a starting viscosity that is higher than the final viscosity after water is added and the resulting end use composition is made.

The hydratable concentrated surfactant composition and end use composition typically have a pH from 3.0 to 6.0.

The end use composition is made by combining water and the hydratable concentrated surfactant composition and mixing (with moderate shear like stirring, preferably shaking) to produce the end use composition.

The compositions of the invention are cosmetic and non-therapeutic.

The end use composition can be a personal care cleaning composition and is preferably a shampoo, make-up wash, facial wash, hand wash or personal care liquid body wash, more preferably a shampoo or a body wash, most preferably a shampoo.

The end use composition is one suitable to be wiped or washed off, and preferably, washed off with water.

In an embodiment of the invention, the end use shampoo composition can preferably have a viscosity from 2,000 to 6,000.

Viscosity, unless noted otherwise, is measured at 30° C. on a Brookfield DV2T using a Spindle RV-05 at 20 rpm for 60 seconds on a Helipath stand.

### Silicone Free

Preferably, the hydratable concentrated surfactant compositions of the invention are free from silicone. In the context of the invention, by free from is meant having less than 0.4 weight %, more preferably less than 0.1 weight %, even more preferably less than 0.05 weight %, still more preferably less than 0.001 weight %, yet preferably less than 0.0001 weight %, and most preferably 0 weight % of silicone by weight of the total composition.

### The Sulphate Free Anionic Surfactant

The hydratable concentrated surfactant composition of the invention includes an anionic surfactant, that is free from sulphate.

Typical sulphate free anionic surfactants for use in the invention include those surface active agents which contain an organic hydrophobic group with from 6 to 22 carbon atoms, preferably from 8 to 22, more preferably from 8 to 18 carbon atoms, even more preferably from 10 to 18 carbon atoms, most preferably 12 to 18 carbon atoms in their molecular structure; and at least one water-solubilising group which is preferably selected from sulphonate, sulphosuccinate, phosphate, sarcosinate, taurate, isethionate, glycinate, glutamate and mixtures thereof, most preferably selected from isothionates, taurates and mixtures thereof.

### Sulphosuccinates

The anionic may also include alkyl sulfosuccinates (including mono- and dialkyl, e.g., C₆-C₂₂ sulfosuccinates); alkyl and acyl taurates (often methyl taurates), alkyl and acyl sarcosinates, sulfoacetates, C₈-C₂₂ alkyl phosphates and phosphonates, alkyl phosphate esters and alkoxyl alkyl phosphate esters, acyl lactates, C₈-C₂₂ monoalkyl succinates and maleates, sulphoacetates, alkyl glucosides and acyl isethionates, and the like.

Sulfosuccinates may be monoalkyl sulfosuccinates having the formula:

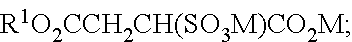

and amide-MEA sulfosuccinates of the formula:

R¹CONHCH₂CH₂O₂CCH₂CH(SO₃M)CO₂M wherein R¹ ranges from C₈-C₂₂ alkyl.

### Sarcosinates

Sarcosinates for use in the compositions of the invention are generally indicated by the formula:

R²CON(CH₃)CH₂CO₂M, wherein R² ranges from C₈-C₂₀ alkyl.

### Isethionates

The isethionates that may be used include C₈-C₁₃ acyl isethionates (including those which have a substituted head group such as a C₁₋₄ alkyl substitution, preferably methyl substitution). These esters are prepared by a reaction between alkali metal isethionate with mixed aliphatic fatty acids having from 6 to 18 carbon atoms and an iodine value of less than 20. Often at least 75% of the mixed fatty acids have from 12 to 18 carbon atoms and up to 25% have from 6 to 10 carbon atoms.

The acyl isethionate used may be an alkoxylated isethionate such as is described in Ilardi et al., U.S. Pat. No. 5,393,466, entitled “Fatty Acid Esters of Polyalkoxylated isethonic acid; issued Feb. 28, 1995; hereby incorporated by reference. This compound has the general formula:

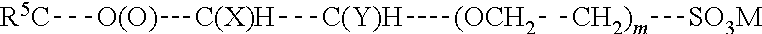

wherein R⁵ is an alkyl group having 8 to 18 carbons, m is an integer from 1 to 4, X and Y are each independently hydrogen or an alkyl group having 1 to 4 carbons and M is a solubilizing cation.

### Taurates

Taurates for use in the hydratable concentrated surfactant composition of the invention are generally identified by formula:

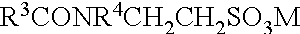

wherein R³ is a C₈-C₂₀ alkyl, R⁴ is a C₁-C₄ alkyl and M is a solubilizing cation.

Suitable taurate surfactants for use in the hydratable concentrated surfactant composition of the invention are acylamides of taurine or N-methyltaurine, and salts thereof, for example, acyl taurates represented by the general formula:

R⁸C(O)N(R⁹)(CH₂)_{y}SO₃M (a),

and preferably by the general formula

R⁸C(O)N(R⁹)CH₂CH₂SO₃M (b),

where R⁸ is C6 to C30, more particularly C6 to C24 alkyl, y is 2 or 3, R⁹ is hydrogen or methyl, and M is a solubilizing cation such as, for example, hydrogen, ammonium, alkali metal cation, a lower, i.e., C to C₄, alkanolammonium cation, or a basic amino acid cation. In one embodiment, R⁸ is C₈ to C₁₈ alkyl. In one embodiment at least half of the R⁸ groups are C8-C18 alkyl. In another embodiment at least half of the R⁸ groups are C10 to C14 alkyl. R⁸ may be saturated or unsaturated. In one embodiment R⁹ is methyl.

Suitable acyl taurates according to formula (a) include, for example, taurates commonly known as sodium methyl lauroyl taurate, potassium methyl lauroyl taurate, sodium methyl myristoyl taurate, potassium methyl myristoyl taurate, ammonium methyl myristoyl taurate, sodium methyl cocoyl taurate, potassium methyl cocoyl taurate, ammonium methyl cocoyl taurate, sodium methyl oleoyl taurate, potassium methyl oleoyl taurate, ammonium methyl oleoyl taurate, sodium lauroyl taurate, potassium lauroyl taurate, ammonium myristoyl taurante, sodium cocoyl taurate, potassium oleoyl taurate, and the like. In one embodiment, a salt of the coconut fatty acid amide of N-methyltaurine is of particular interest.

### Sulphonates

Anionic surfactants suitable for use in the hydratable concentrated surfactant composition of the present invention, include aliphatic sulfonates, such as a primary alkane (e.g., C₈-C₂₂) sulfonate, primary alkane (e.g., C₈-C₂₂) disulfonate, C₈-C₂₂ alkene sulfonate, C₈-C₂₂ hydroxyalkane sulfonate or alkyl glyceryl ether sulfonate (AGS); or aromatic sulfonates such as alkyl benzene sulfonate.

A preferred sulphonate surfactant is alpha olefin sulphonate. Alpha olefin sulfonate anionic surfactants for use in the present invention preferably have the general formula (I)

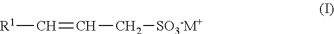

in which R¹ is selected from linear or branched alkyl groups having from 11 to 13 carbon atoms and mixtures thereof; and M is a solubilizing cation;

Preferably R¹ in general formula (I) is a C₁₄ or C₁₆ linear alkyl group.

Preferably M in general formula (I) is selected from alkali metal cations (such as sodium or potassium), ammonium cations and substituted ammonium cations (such as alkylammonium, alkanolammonium or glucammonium).

Commercially produced alpha olefin sulfonate anionic surfactants of general formula (I) may be made by sulfating C14-16 olefins derived from natural gas. The process can also yield mixtures of homologues and low levels of unreacted olefins.

Particularly preferred is alpha olefin sulfonate with an average of 14-16 carbons. A suitable example of such a material is Bioterge AS40 (ex Stepan).

### Glycinates and Glutamates

The sulphate free anionic surfactant may be a glycinate surfactant ora glutamate surfactant.

Preferred glycinates are sodium lauroyl glycinate and sodium cocoyl glycinate.

Preferred glutamates are sodium lauroyl glutamate and sodium cocoyl glutamate.

In an embodiment of the invention, an anionic surfactant used is selected from sodium lauroyl glycinate, sodium cocoyl glycinate, sodium lauroyl glutamate, sodium cocoyl glutamate, sodium lauroyl isethionate, sodium cocoyl isethionate, sodium methyl lauroyl taurate, sodium methyl cocoyl taurate or a mixture thereof. Such anionic surfactants are commercially available from suppliers like Galaxy Surfactants, Clariant, Sino Lion and Innospec. Sodium cocoyl isethionate, sodium methyl lauroyl taurate, sodium lauroyl glyconate, sodium methyl lauroyl isethionate or mixtures thereof are the preferred anionics suitable for use.

Specific examples of suitable anionic surfactants include, sodium lauryl sarcosinate, sodium lauroyl sarcosinate, lauryl sarcosine, sodium tridecyl benzene sulphonate, sodium dodecyl benzene sulphonate, sodium methyl cocoyl taurate sodium cocoyl isethionate and mixtures thereof. Preferably the anionic surfactant is selected from sodium methyl cocoyl taurate and sodium cocoyl isethionate, most preferably sodium methyl cocoyl taurate.

Mixtures of any of the above described materials may also be used.

In a typical hydratable concentrated surfactant composition of the invention the level of anionic surfactant will generally range from 5 to 40%, preferably from 7 to 35, more preferably from 10 to 30, most preferably from 15 to 30% (by weight based on the total weight of the composition and 100% active level).

The Amphoteric and/or Zwitterionic Surfactant

The hydratable concentrated surfactant composition of the invention includes a co-surfactant, which is an amphoteric or zwitterionic surfactant. Preferably, the amphoteric and/or zwitterionic surfactant is selected from a betaine, an amphoacetate, a sultaine and mixtures thereof.

The level of co-surfactant is generally from 5 to 40%, preferably from 7 to 35, more preferably from 10 to 30, most preferably from 15 to 30% by weight based on the total weight of the hydratable concentrated surfactant composition and based on 100% active level.

Preferably the co-surfactant is an amphoteric surfactant. Suitable amphoteric surfactants are betaines, such as those having the general formula R(CH₃)₂N⁺CH₂COO—, where R is an alkyl or alkylamidoalkyl group, the alkyl group preferably having from 6 to 22 carbon atoms, more preferably from 8 to 22 carbon atoms, even more preferably form 8 to 18 carbon atoms, still more preferably from 10 to 18 carbon atoms, most preferably from 12 to 18 carbon atoms, and mixtures thereof.

### Betaine Surfactant

Betaines that are suitable for use in the present invention can be represented by the general formula:

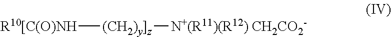

where R¹⁰ is C6 to C30, more particularly C6 to C24 alkyl, z is 0 or 1, R¹¹ and R¹² are independently alkyl, hydroxyalkyl or carboxyalkyl of 1 to 3 carbon atoms, and y is 2 or 3; and salts thereof. In one embodiment, at half of the groups R¹⁰ are C8 to C18 alkyl. In another embodiment, at least half of the groups R¹⁰ are C 10 to C14 alkyl. R¹⁰ may be saturated or unsaturated. In one embodiment, R¹⁰is derived from coconut oil or palm kernel oil. In one embodiment R¹¹ and R¹² are methyl.

The formula (IV) betaines include the simple betaines:

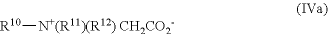

where R¹⁰, R¹¹, and R¹² are as described above, and the amidobetaines:

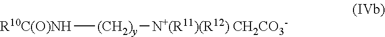

where R¹⁰, R¹¹, R¹², and y are as described above.

Particularly suitable betaines are oleyl betaine, caprylamidopropyl betaine, lauramidopropyl betaine, isostearylamidopropyl betaine, and cocoamidopropyl betaine and mixtures thereof. Most preferably, the co-surfactant is cocamidopropyl betaine.

### Zwitterionic

Zwitterionic surfactants that are suitable for use in the present invention, include at least one acid group. Such an acid group may be a carboxylic or a sulphonic acid group. They often include quaternary nitrogen, and therefore, can be quaternary amino acids. They should generally include an alkyl or alkenyl group with from 6 to 22 carbon atoms, more preferably from 8 to 22 carbon atoms, even more preferably from 8 to 18 carbon atoms, still more preferably from 10 to 18 carbon atoms, most preferably from 12 to 18 carbon atoms, and mixtures thereof. These surfactants will generally comply with an overall structural formula:

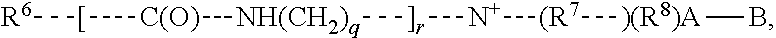

where R⁷ is alkyl or alkenyl of from 6 to 22, preferably 8 to 22, more preferably 8 to 18 carbon atoms, still more preferably from 10 to 18 carbon atoms, even more preferably 12 to 18 carbon atoms; R⁷ and R⁸ are each independently alkyl, hydroxyalkyl or carboxyalkyl of 1 to 3 carbon atoms; q is 2 to 4; r is 0 to 1; A is alkylene of 1 to 3 carbon atoms optionally substituted with hydroxyl, and B is —CO₂— or —SO₃—.

### Amphoacetates

Zwitterionic surfactants suitable for use in the invention include sodium acyl amphoacetates, sodium acyl amphopropionates, disodium acyl amphodiacetates and disodium acyl amphodipropionates where the acyl (i.e., alkanoyl group) can comprise a alkyl portion with from 6 to 22 carbon atoms, more preferably from 8 to 22 carbon atoms, even more preferably form 8 to 18 carbon atoms, still more preferably from 10 to 18 carbon atoms, most preferably from 12 to 18 carbon atoms, and mixtures thereof. Illustrative examples of the amphoteric surfactants suitable for use include sodium lauroamphoacetate, sodium cocoamphoacetate, sodium lauroamphoacetate, sodium cocoamphoacetate and mixtures thereof.

### Sultaines

A zwitterionic surfactant suitable for use is cocamidopropyl sultaine. Such surfactants are made commercially available from suppliers like Stepan Company, and it is within the scope of the invention to employ mixtures of the aforementioned surfactants.

The weight ratio of a) the sulphate free anionic surfactant to b) the amphoteric and/or zwitterionic surfactant is from 1:1 to 1:2, preferably 1:1.4.

The composition preferably comprises a total amount of anionic surfactant (a) and amphoteric and/or zwitterionic surfactant (b) of at least 20 wt %; more preferably at least 22 wt %, even more preferably at least 24 wt % and most preferably at least 25 wt %.

### The First Viscosity Modifier

The hydratable concentrated surfactant composition of the invention comprises a first viscosity modifier, which acts to thicken the diluted end product.

The first viscosity modifier is selected from electrolytes, polymeric thickeners, ethoxylated fatty acid esters, amines comprising 12 to 18 carbon atoms and mixtures thereof.

### Electrolyte Thickeners

A preferred electrolyte thickener is an inorganic electrolyte. Suitable inorganic electrolytes for use in the invention include metal chlorides (such as sodium chloride, potassium chloride, calcium chloride, magnesium chloride, zinc chloride, ferric chloride and aluminum chloride) and metal sulphates (such as sodium sulphate and magnesium sulphate). The inorganic electrolyte is used to provide viscosity to the composition.

Examples of preferred inorganic electrolytes for use in the invention include sodium chloride, potassium chloride, magnesium sulphate and mixtures thereof.

Mixtures of any of the above described materials may also be suitable.

When included, the level of inorganic electrolyte in compositions of the invention generally ranges from about 0.1 to 6%, preferably from about 0.25 to 5% (by total weight of the hydratable concentrated surfactant composition).

### Polymeric Thickeners

Preferred polymeric thickeners are selected from polysaccharides, starches, cellulosic materials (for example fibres such as citrus microfibrils) and mixtures thereof.

### Polysaccharides

Preferred polysaccharides are guar gums, xanthan gums and carrageenans.

Suitable gums include xanthan, sclerotium, pectin, karaya, arabic, agar, guar (including Acacia senegal guar), carrageenan, alginate and combinations thereof.

### Starches

Representative of the starches are chemically modified starches such as sodium hydroxypropyl starch phosphate and aluminum starch octenylsuccinate. Tapioca starch is often preferred, as is maltodextrin.

### Cellulosic Materials

Suitable cellulosic materials include hydroxypropyl cellulose, hydroxypropyl methylcellulose, ethylcellulose, sodium carboxy methylcellulose (cellulose gum/carboxymethyl cellulose) and cellulose (e.g. cellulose microfibrils, cellulose nanocrystals or microcrystalline cellulose).

Sources of cellulose microfibrils include secondary cell wall materials (e.g. wood pulp, cotton), bacterial cellulose, and primary cell wall materials. Preferably the source of primary cell wall material is selected from parenchymal tissue from fruits, roots, bulbs, tubers, seeds, leaves and combination thereof; more preferably is selected from citrus fruit, tomato fruit, peach fruit, pumpkin fruit, kiwi fruit, apple fruit, mango fruit, sugar beet, beet root, turnip, parsnip, maize, oat, wheat, peas and combinations thereof; and even more preferably is selected from citrus fruit, tomato fruit and combinations thereof. A most preferred source of primary cell wall material is parenchymal tissue from citrus fruit. Citrus fibers, such as those made available by Herbacel® as AQ Plus can also be used as source for cellulose microfibrils. The cellulose sources can be surface modified by any of the known methods including those described in Colloidal Polymer Science, Kalia et al., “Nanofibrillated cellulose: surface modification and potential applications” (2014), Vol 292, Pages 5-31.

### Ethoxylated Fatty Acid Esters

High molecular weight ethoxylated fatty acid esters may be used. Illustrative examples include PEG 120 methyl glucose dioleate, PEG 18 glyceryloleate/cocoate, PEG 150 pentaerythritol tetrastearate, mixtures thereof or the like. A preferred polymeric viscosity aid is PEG 150 pentaerythritol tetrastearate which is sold under the Versathix name by Croda.

### Amines

A preferred amine comprising C12 to C18 carbon atoms is Stearamidopropyl Dimethylamine (TAS).

The first viscosity modifier is present in an amount of from 0.01 to 5 wt %, preferably from 0.1 to 4 wt %, more preferably, from 0.1 to 3%, most preferably 0.5 to 2 wt %, by weight of the concentrated hydratable composition.

The first viscosity modifier is present in an amount of from from 0.01 to 1 wt %, preferably from 0.01 to 0.8 wt %, more preferably, from 0.1 to 0.5%, and most preferably, from 0.15 to 0.3% by weight of the diluted end composition.

Second viscosity modifier

The hydratable concentrated surfactant composition of the invention comprises a second viscosity modifier, which acts to lower viscosity of the hydratable concentrated surfactant composition, prior to dilution with water.

Any polyol that lowers viscosity of the hydratable concentrated surfactant composition, for example polypropylene glycol (PPG), polyethylene glycol, monopropylene glycol (MPG) and glycerine.

These are generally polyhydric alcohol type materials. Typical polyhydric alcohols include glycerol (also known as glycerine or glycerin), propylene glycol, dipropylene glycol, polypropylene glycol (e.g., PPG-9), polyethylene glycol, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof. Most preferred polyols are selected from glycerol, polyethylene glycol, propylene glycol and a mixture thereof. The amount of polyol employed may range anywhere from 0.1 to 15% by weight of the total weight of the compositions, preferably from 0.5 to 10%, and more preferably, from 0.5 to 8% by weight of the total weight of the end use composition.

### Form of Product

The hydratable concentrated surfactant composition is preferably a lamellar composition. The composition transforms from lamellar to isotropic form upon dilution to form the end use product.

### Optional Nonionic Surfactant

Nonionic surfactants may optionally be used in the hydratable concentrated surfactant composition and end use composition of the present invention. When used, nonionic surfactants are typically used at levels of from 0.5 to 12 wt %, preferably from 1 to 10 wt %, more preferably from 1.5 to 8 wt %, most preferably from 2 wt % to 6 wt %, by weight of the end use composition.

The nonionic surfactants which may be used include in particular the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic surfactant compounds are alkyl (C₆-C₂₂) phenols ethylene oxide condensates, the condensation products of aliphatic (C₈-C₁) primary or secondary linear or branched alcohols with ethylene oxide, and products made by condensation of ethylene oxide with the reaction products of propylene oxide and ethylenediamine. Other nonionic surfactants include long chain tertiary amine oxides, long chain tertiary phosphine oxides, dialkyl sulphoxides, and the like.

In an embodiment of the invention, nonionic surfactants optionally used can include fatty acid/alcohol ethoxylates having the following structures a) HOCH₂(CH₂)ₛ(CH₂CH₂O)ᵥH or b) HOOC(CH₂)_{c}(CH₂CH₂O)_{d} H; where s and v are each independently an integer up to 18; and c and d are each independently an integer from 1 or greater. In an embodiment of the invention, s and v are each independently 6 to 18; c and d are each independently 1 to 30. Other options for nonionic surfactants include those having the formula HOOC(CH₂)—CH═CH—(CH₂)ₖ(CH₂CH₂O)_{z} H, where i, k are each independently 5 to 15; and z is 5 to 50.

In another embodiment of the invention, i and k are each independently 6 to 12; and z is 15 to 35.

The nonionic surfactant may also include a sugar amide, such as a polysaccharide amide. Specifically, the surfactant may be one of the lactobionamides described in U.S. Pat. No. 5,389,279 to Au et al., entitled “Compositions Comprising Nonionic Glycolipid Surfactants issued Feb. 14, 1995; which is hereby incorporated by reference or it may be one of the sugar amides described in U.S. Pat. No. 5,009,814 to Kelkenberg, titled “Use of N-Poly Hydroxyalkyl Fatty Acid Amides as Thickening Agents for Liquid Aqueous Surfactant Systems” issued Apr. 23, 1991; hereby incorporated into the subject application by reference.

### Optional Cationic Surfactant

In an embodiment of the invention, cationic surfactants may optionally be used in the hydratable concentrated surfactant composition and end use composition of the present invention.

One class of optional cationic surfactants includes heterocyclic ammonium salts such as cetyl or stearyl pyridinium chloride, alkyl amidoethyl pyrrylinodium methyl sulfate, and lapyrium chloride.

Tetra alkyl ammonium salts are another useful class of cationic surfactants suitable for optional use. Examples include cetyl or stearyl trimethyl ammonium chloride or bromide; hydrogenated palm or tallow trimethylammonium halides; behenyl trimethyl ammonium halides or methyl sulfates; decyl isononyl dimethyl ammonium halides; ditallow (or distearyl) dimethyl ammonium halides, and behenyl dimethyl ammonium chloride, preferably cetyl trimethyl ammonium chloride (CTAC).

Still other types of cationic surfactants that may be used are the various ethoxylated quatemary amines and ester quats. Examples include PEG-5 stearyl ammonium lactate (e.g., Genamin KSL manufactured by Clariant), PEG-2 coco ammonium chloride, PEG-15 hydrogenated tallow ammonium chloride, PEG 15 stearyl ammonium chloride, dipalmitoyl ethyl methyl ammonium chloride, dipalmitoyl hydroxyethyl methyl sulfate, and strearyl amidopropyl dimethylamine lactate.

Even other useful cationic surfactants suitable for optional use include quaternized hydrolysates of silk, wheat, and keratin proteins, and it is within the scope of the invention to use mixtures of the aforementioned cationic surfactants.

If used, cationic surfactants will make up no more than 1.0% by weight of the hydratable composition. When present, they typically make up from 0.01 to 0.7%, and more typically, from 0.1 to 0.5% by weight of the end use composition, including all ranges subsumed therein.

### Water

Water makes up from 10 to 70 wt %, preferably 10 to 65 wt %, more preferably 10 to 60 wt %, even preferably 10 to 40%, still more preferably from 10 to 30%, most preferably from 12 to 30 wt % by weight based on total weight of the hydratable concentrated surfactant composition.

Alternatively, water can be replaced with a mixture of water and polyol, preferably glycerol.

pH

The pH of the hydratable composition and end use composition is typically from 3 to 6, preferably from 3.5 to 5.5, more preferably from 3.5 to 5, most preferably from 3.8 to 4.8. Adjusters suitable to modify/buffer the pH may be used. Such pH adjusters include triethylamine, NaOH, KOH, H₂SO₄, HCl, C₆ H₈ O₇ (i.e., citric acid) or mixtures thereof. The pH adjusters are added at amounts to yield the desired final pH. The pH values may be assessed with commercial instrumentation such as a pH meter made commercially available from Thermo Scientific®.

### Preservatives

Preservatives are used in the hydratable concentrated surfactant composition and end use composition to protect against the growth of potentially harmful microorganisms. Cosmetic chemists are familiar with appropriate preservatives and routinely choose them to satisfy the preservative challenge test and to provide product stability. Suitable traditional preservatives for use include hydantoin derivatives and propionate salts. Particularly preferred preservatives are iodopropynyl butyl carbamate, phenoxyethanol, 1,2-octanediol, hydroxyacetophenone, ethylhexylglycerine, hexylene glycol, methyl paraben, propyl paraben, imidazolidinyl urea, sodium dehydroacetate, dimethyl-dimethyl (DMDM) hydantoin and benzyl alcohol and mixtures thereof. Other preservatives suitable for use include sodium dehydroacetate, chlorophenesin and decylene glycol. The preservatives should be selected having regard for the use of the composition and possible incompatibilities between the preservatives and other ingredients in the emulsion. Preservatives are preferably employed in amounts ranging from 0.01% to 2.0% by weight of the total weight of the end use composition (up to 7% by weight of total hydratable composition), including all ranges subsumed therein.

Preferably, the preservative comprises an alkali metal salt of benzoic acid and a metal ion sequestrant (preferably EDTA).

### Optional Wet Feel Polymers

Cationic polymers are preferred ingredients in a compositions of the invention for enhancing conditioning performance.

Suitable cationic polymers may be homopolymers which are cationically substituted or may be formed from two or more types of monomers. The weight average (M_{W}) molecular weight of the polymers will generally be between 100 000 and 3 million daltons. The polymers will have cationic nitrogen containing groups such as quaternary ammonium or protonated amino groups, or a mixture thereof. If the molecular weight of the polymer is too low, then the conditioning effect is poor. If too high, then there may be problems of high extensional viscosity leading to stringiness of the composition when it is poured.

The cationic nitrogen-containing group will generally be present as a substituent on a fraction of the total monomer units of the cationic polymer. Thus when the polymer is not a homopolymer it can contain spacer non-cationic monomer units. Such polymers are described in the CTFA Cosmetic Ingredient Directory, 3rd edition. The ratio of the cationic to non-cationic monomer units is selected to give polymers having a cationic charge density in the required range, which is generally from 0.2 to 3.0 meq/gm. The cationic charge density of the polymer is suitably determined via the Kjeldahl method as described in the US Pharmacopoeia under chemical tests for nitrogen determination.

Suitable cationic polymers include, for example, copolymers of vinyl monomers having cationic amine or quaternary ammonium functionalities with water soluble spacer monomers such as (meth)acrylamide, alkyl and dialkyl (meth)acrylamides, alkyl (meth)acrylate, vinyl caprolactone and vinyl pyrrolidine. The alkyl and dialkyl substituted monomers preferably have C1-C7 alkyl groups, more preferably C1-3 alkyl groups. Other suitable spacers include vinyl esters, vinyl alcohol, maleic anhydride, propylene glycol and ethylene glycol.

The cationic amines can be primary, secondary or tertiary amines, depending upon the particular species and the pH of the composition. In general, secondary and tertiary amines, especially tertiary, are preferred.

Amine substituted vinyl monomers and amines can be polymerised in the amine form and then converted to ammonium by quaternization.

The cationic polymers can comprise mixtures of monomer units derived from amine- and/or quaternary ammonium-substituted monomer and/or compatible spacer monomers.

Suitable (non-limiting examples of) cationic polymers include:
cationic diallyl quaternary ammonium-containing polymers including, for example, dimethyldiallylammonium chloride homopolymer and copolymers of acrylamide and dimethyldiallylammonium chloride, referred to in the industry (CTFA) as Polyquaternium 6 and Polyquaternium 7, respectively;
mineral acid salts of amino-alkyl esters of homo-and co-polymers of unsaturated carboxylic acids having from 3 to 5 carbon atoms, (as described in U.S. Pat. No. 4,009,256);
cationic polyacrylamides(as described in WO95/22311).

Other cationic polymers that can be used include cationic polysaccharide polymers, such as cationic cellulose derivatives, cationic starch derivatives, and cationic guar gum derivatives. Cationic polysaccharide polymers suitable for use in compositions for use in the invention include monomers of the formula:

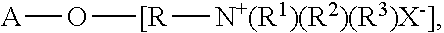

wherein: A is an anhydroglucose residual group, such as a starch or cellulose anhydroglucose residual. R is an alkylene, oxyalkylene, polyoxyalkylene, or hydroxyalkylene group, or combination thereof. R¹, R² and R³ independently represent alkyl, aryl, alkylaryl, arylalkyl, alkoxyalkyl, or alkoxyaryl groups, each group containing up to about 18 carbon atoms. The total number of carbon atoms for each cationic moiety (i.e., the sum of carbon atoms in R¹, R² and R³) is preferably about 20 or less, and X is an anionic counterion.

Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from the Amerchol Corporation, for instance under the tradename Polymer LM-200.

Other suitable cationic polysaccharide polymers include quaternary nitrogen-containing cellulose ethers (e.g. as described in U.S. Pat. No. 3,962,418), and copolymers of etherified cellulose and starch (e.g. as described in U.S. Pat. No. 3,958,581). Examples of such materials include the polymer LR and JR series from Dow, generally referred to in the industry (CTFA) as Polyquaternium 10.

A particularly suitable type of cationic polysaccharide polymer that can be used is a cationic guar gum derivative, such as guar hydroxypropyltrimethylammonium chloride (commercially available from Rhodia in their JAGUAR trademark series). Examples of such materials are JAGUAR C₁₃S, JAGUAR C14 and JAGUAR C₁₇.

Mixtures of any of the above cationic polymers may be used.

Cationic polymer will generally be present in a shampoo composition for use in the invention at levels of from 0.01 to 5%, preferably from 0.02 to 1%, more preferably from 0.05 to 0.8% by total weight of cationic polymer based on the total weight of the composition.

### Other Ingredients

Fragrances, fixatives, chelators and exfoliants may optionally be included in the compositions of the present invention. Each of these substances may range from about 0.03 to about 5%, preferably between 0.1 and 3% by weight of the total weight of the end use composition, including all ranges subsumed therein. To the extent the exfoliants are used, those selected should be of small enough particle size so that they do not impede the performance of any packaging used to dispense the compositions of this invention. Conventional emulsifiers having an HLB of greater than 8 may optionally be used. Illustrative examples include Tween, 40, 60, 80, polysorbate 20 and mixtures thereof.

Typically, emulsifiers for water continuous systems make up from 0.3 to 2.5% by weight of the end use composition.

Unless indicated otherwise, as used herein, “%” means weight %, alternatively referred to as % by weight. All references to the amount by weight of a component of the instant composition are, unless indicated otherwise, based on the total weight of the composition. Unless indicated otherwise, all ratios are by weight.

All amounts referred to herein are based on 100% activity (or “active”) unless otherwise stated. By 100% activity (or “active”) is meant that the material is not diluted and is at 100% v/v or wt/wt. Many materials used in personal care formulations are commercially available at different active concentrations, for example at 70% active or 60% active. For example, 100 ml of 70% active surfactant provides the same amount of active material as 70 ml of 100% active surfactant. Therefore, in order to provide for variations in activities of materials, all amounts are given based on 100% active materials, unless otherwise stated.

All numerical ranges employed in this description ought to be understood as modified by the word “about”. Numerical ranges are understood to encompass the ranges expressly disclosed, as well as ranges subsumed by same. Where the system or method of the subject invention is described as “including” or “comprising” specific components and/or features, narrower embodiments that “consist essentially of” or “consist of” the recited components and/or features are also contemplated.

The Examples provided are to facilitate an understanding of the invention. They are not intended to limit the scope of the claims.

### EXAMPLES

### Example 1: Compositions 1-3 in Accordance with the Invention and Comparative Compositions A and B

Concentrated shampoo compositions 1-3 were prepared in accordance with the invention.

A comparative dilute shampoo composition A was also prepared.

Comparative concentrated shampoo, B, was the same as composition 3 but prepared without the inclusion of a poiyoi.

The compositions of Compositions 1-3, in accordance with the invention, and Comparative composition A are shown in Table 1 below.

TABLE 1
Composition of Compositions 1-3 in accordance with
the invention and Comparative compositions A and B

| | % | | | | | |
|---|---|---|---|---|---|---|
| | Activity | Concentrate | | | | |
| | of Raw | A | B | 1 | 2 | 3 |
| Ingredient | Material | % Active Inclusion level as 100% | | | | |
| Water | 100 | to | to | to | to | to |
| | | 100 | 100 | 100 | 100 | 100 |
| Cocamidopropyl | 82 | 4.75 | 23.75 | 14.25 | 19 | 23.75 |
| Betaine | | | | | | |
| Sodium Methyl | 70 | 3.4 | 17 | 10.2 | 13.6 | 17 |
| Cocoyl Taurate | | | | | | |
| Fragrance | 100 | 0.7 | 3.5 | 2.1 | 2.8 | 3.5 |
| Disodium | 100 | 0.05 | 0.25 | 0.25 | 0.25 | 0.25 |
| Ethylene Diamine | | | | | | |
| Tetraacetic acid | | | | | | |
| Sodium Benzoate | 100 | 0.5 | 2.5 | 2.5 | 2.5 | 2.5 |
| Citric Acid | 100 | 0.5 | 2.5 | 1.5 | 2 | 2.5 |
| Stearamidopropyl | 100 | 0.1 | 0.5 | 0.3 | 0.4 | 0.5 |
| Dimethylamine | | | | | | |
| Sodium Chloride | 100 | 0.4 | 2 | 2 | 2 | 2 |
| Glycerine | 100 | 0 | 0 | 2.4 | 3.2 | 4 |
| pH | | 4.2 | 4.2 | 4.5 | 4.1 | 4.2 |

Concentrated compositions 1-3 were in the form of a thick paste.

Comparative Example A was a liquid.

During preparation, Comparative Example B formed a solid lumpy mass on the stirrer, which made processing very difficult.

Concentrated shampoo compositions 1-3, and Comparative compositions A and B were prepared by the following method:
Stearamidopropyl dimethylamine was dissolved in hot water.
Sodium methyl cocoyl taurate was then added at 65° C. and mixed under constant shear.
Upon cooling to 50° C., cocamidopropyl betaine was added and all ingredients thoroughly mixed until homogeneous.
In a side pot to a 10-20% portion of the total water at less than 50° C. was added sodium benzoate, sodium chloride and ethylenediaminetetraacetic acid together with glycerol.
The temperature was cooled to less than 40° C. and fragrance was added with stirring.
Finally, citric acid was added to adjust pH.

### Example 2: Dilution of Concentrated Compositions 1-3 and B

Concentrated compositions 1-3 and B were diluted for use as dilute shampoos are follows:

Method for Preparing an End Use Diluted Product from Concentrated Compositions 1-3

In the method the following amounts of concentrate to water were used to make 500 g of the end use product:
Composition 1: 1 part concentrate to 2 parts water
Composition 2: 1 part concentrate to 3 parts water
Composition 3 and B: 1 part concentrate to 4 parts water
1. Weigh out the required amount of concentrate formulation into a clear container.
2. Boil water and cool down until temperature reaches 50° C.
3. Add the correct amount of cooled boiled water to the container.
4. Seal container and shake for 30 seconds.
5. Leave to stand and evaluate undissolved parts every minute.
6. At 5 minutes evaluate again and shake container again for 30 seconds if required.

Composition B dissolved at a very slow rate making it unsuitable for use as a concentrated product that needs to be diluted at point of use.

### Example 3: Viscosities of Compositions 1-3 and Comparative Compositions A and B

The viscosities of Compositions 1-3 and Comparatives A and B were measured in concentrated (pre-diluted) form and diluted forms.

The viscosities are given in Table 2 below:

TABLE 2
viscosities of Compositions 1-3 and
comparative Compositions A and B

| Concentrate | A | B | 1 | 2 | 3 |
|---|---|---|---|---|---|
| Viscosity | — | 750000* | 6000** | 10000** | 300000* |
| Concentrate (cPs) | | | | | |
| Viscosity Dilute | 4000** | — | 3620** | 4360** | 2501** |
| (cPs) | | | | | |

*Viscosity is measured at 30° C. on a Brookfield DV2T using T-Bar B at 0.5 rpm for 60 seconds on a Helipath stand
**Viscosity is measured at 30° C. on a Brookfield DV2T using a Spindle RV-05 at 20 rpm for 60 seconds on a Helipath stand

It will be seen that the viscosities of the diluted compositions are consistent with a standard shampoo. This is acceptable to the consumer, who prefers a thick dilute.

Viscosity cues sensory performance, unlike a thin product. This has not been achieved before.

It was observed that Composition B was unacceptably thick and solid and could not be diluted at point of use.

1. A hydratable concentrated surfactant composition comprising, based on the total weight of the composition and 100% activity:
a) from 5 wt % to 40 wt % of a sulphate free anionic surfactant comprising 6 to 22 carbon atoms;
b) from 5 wt % to 40 wt % of an amphoteric and/or zwitterionic surfactant;
c) from 0.01 wt % to 5 wt % of a first viscosity modifier, selected from electrolytes, polymeric thickeners, ethoxylated fatty acid esters, amines comprising from 12 to 18 carbon atoms and mixtures thereof,
d) from 0.1 wt % to 15 wt % of a second viscosity modifier, which is a polyol;
e) a preservative; and
f) from 10 wt % to 70 wt % water;
wherein the hydratable concentrated surfactant composition has a pH of from 3 to 6; and
wherein the hydratable concentrated surfactant composition has a viscosity of from 6000 to 400,000 cps, when measured at 30° C. on a Brookfield DV2T using a Spindle RV-05 at 20 rpm for 60 seconds on a Helipath stand.

2. The hydratable concentrated surfactant composition of claim 1, wherein the composition comprises a total amount of anionic surfactant (a) and amphoteric and/or zwitterionic surfactant (b) of at least 20 wt %.

3. The hydratable concentrated surfactant composition of claim 1, wherein the sulphate free anionic surfactant comprises an organic hydrophobic group with from 6 to 22 carbon atoms, carbon atoms; and at least one water-solubilising group which is selected from sulphonate, sulphosuccinate, phosphate, sarcosinate, taurate, isethionate, glycinate, glutamate and mixtures thereof.

4. The hydratable concentrated surfactant composition of claim 1, wherein the amphoteric and/or zwitterionic surfactant is selected from a betaine, an amphoacetate, a sultaine and mixtures thereof.

5. The hydratable concentrated surfactant composition of claim 1, wherein the amphoteric and/or zwitterionic surfactant is present in an amount of from 7 wt % to 35 wt %.

6. The hydratable concentrated surfactant composition of claim 1, wherein the first viscosity modifier is a polymeric thickener selected from polysaccharides, starches, cellulosic materials and mixtures thereof.

7. The hydratable concentrated surfactant composition of claim 1, wherein the second viscosity modifier is a polyhydric alcohol selected from glycerol, propylene glycol, dipropylene glycol, polypropylene glycol, polyethylene glycol, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof.

8. The hydratable concentrated surfactant composition of claim 1, wherein the weight ratio of a) to b) is from 1:1 to 1:2.

9. The hydratable concentrated surfactant composition of claim 1, wherein the composition is a lamellar composition.

10. The hydratable concentrated surfactant composition of claim 1, wherein the composition transforms from lamellar to isotropic form upon dilution.

11. An end use composition prepared by hydrating the hydratable concentrated surfactant composition of claim 1 by dilution with water.

12. The end use composition of claim 11 wherein a hydratable concentrated surfactant composition to water weight ratio of from 1:1 to 1:6 is used.

13. The end use composition of claim 11, wherein the composition has a viscosity of from 2000 to 10,000 cPs when measured at 30° C. on a Brookfield DV2T using a Spindle RV-05 at 20 rpm for 60 seconds on a Helipath stand.

14. A method of preparing an end use composition, the method comprising the step of diluting the hydratable concentrated surfactant composition of claim 1 with water.

15. The method of claim 14, wherein the water has a temperature of from 10 to 50° C., the method comprises a step of applying moderate shear such as shaking or stirring to the mixture of hydratable concentrated surfactant composition and water to yield the end use composition in less than 5 minutes.

16. The hydratable concentrated surfactant composition of claim 1, wherein the weight ratio of a) to b) is 1:1.4.

17. The end use composition of claim 11, wherein a hydratable concentrated surfactant composition to water weight ratio of from 1:1 to 1:5 is used.

18. The end use composition of claim 11, wherein the composition has a viscosity of from 2000 to 7500 cPs when measured at 30° C. on a Brookfield DV2T using a Spindle RV-05 at 20 rpm for 60 seconds on a Helipath stand.

19. The method of claim 14, wherein the water has a temperature of from 10 to 50° C., the method comprises a step of applying moderate shear such as shaking or stirring to the mixture of hydratable concentrated surfactant composition and water to yield the end use composition in less than 3 minutes.

20. The method of claim 14, wherein the water has a temperature of from 10 to 50° C., the method comprises a step of applying moderate shear such as shaking or stirring to the mixture of hydratable concentrated surfactant composition and water to yield the end use composition in less than 2 minutes.
